(19) United States
(12) Patent Application Publication
(10) Pub. No.: US 2004/0214181 A1
(43) Pub. Date: Oct. 28, 2004

(54) Knockout reagent surrogate screening assay

(76) Inventors: Vic Evan Myer, Harvard, MA (US); Arthur John Kudla, Malden, MA (US)

Correspondence Address: CLARK & ELBING LLP, 101 FEDERAL STREET, BOSTON, MA, 02110, US

(21) Appl. No.: 10/424,053

(22) Filed: Apr. 25, 2003

(51) Int. Cl. C12Q 1/68

(52) U.S. Cl. 435/6

(57) ABSTRACT

The present invention features a method and array for high throughput analysis of candidate knockout reagents in order to identify those capable of gene silencing.

### BACKGROUND OF THE INVENTION

RNA interference (RNAi) is a method for decreasing the cellular expression of specific proteins of interest (reviewed in Tuschl (2001) Chembiochem 2:239-245; Sharp (2001) Genes & Devel. 15:485-490; Hutvagner and Zamore (2002) Curr. Opin. Genet. Devel. 12:225-232; Hannon (2002) Nature 418:244-251). In RNAi, gene silencing is typically triggered post-transcriptionally by the presence of double-stranded RNA (dsRNA) in a cell. This dsRNA is processed intracellularly into shorter pieces called small interfering RNAs (siRNAs). The introduction of siRNAs into cells either by transfection of dsRNAs or through expression of siRNAs using a plasmid-based expression system is increasingly being used to create loss-of-function phenotypes in mammalian cells.

A significant challenge in the field of knockout biology is to devise a method of screening sequences to determine their functionality in vivo. To determine the functionality of a given sequence, practitioners currently utilize assays based on examination of gene-specific effects or assays that require significant effort, are costly, and not amenable to scale-up. An example of the first type of assay is one utilizing an antibody that binds a protein of interest. Such an assay typically requires an antibody that is specific for the target protein of interest. An example of the second type of assay is real-time PCR on RNA isolated from cells transfected with a knockout reagent. To do this assay, a primer-probe pair must be generated and quality controlled for each gene that is being knocked down. After generation of this reagent, the researcher transfects cells with the reagent, isolates RNA from those cells, quality controls the RNA and performs real-time PCR. The approach is constrained by the requirements for generation of the quality controlled primer-probe sets and performance of the assay itself. Assays for identifying active forms of other knockout reagents (e.g., antisense RNAs, ribozymes, triple helix forming oligonucleotides) suffer from similar deficiencies.

Thus, there is a need for improved assays for identifying active knockout reagents.

### SUMMARY OF THE INVENTION

The present invention features a method and array for high throughput analysis of candidate knockout reagents in order to identify those capable of gene silencing.

Accordingly, the invention features method for identifying a nucleic acid molecule capable of gene silencing. In this method, a plurality of nucleic acid molecules are deposited onto a surface in discrete, defined locations. In particular, at each location is deposited a plurality of first nucleic acid molecules, wherein the first nucleic acid molecules include candidate knockout reagents (e.g., double-stranded RNA molecules, ribozymes, antisense nucleic acid molecules, or triple helix forming oligonucleotides) or encode candidate knockout reagents, and a plurality of second nucleic acid molecules, wherein each second nucleic acid molecule includes (i) a promoter; (ii) a reporter gene having a 5′ and/or 3′ untranslated region, the reporter gene being operably linked to the promoter for expression in a cell; and (iii) a target nucleic acid derived from the target gene. The target nucleic acid is located within either untranslated region. Different first nucleic acid molecules are deposited at different discrete, defined locations. If desirable, pools of two or more different candidate knockout reagents can be deposited at a given location. The deposited nucleic acid molecules are then contacted with cells under appropriate conditions for entry of the nucleic acid molecules into the cells. The nucleic acid molecules are introduced into the cells at the location in which each of the nucleic acid molecules was deposited. Following introduction of the nucleic acid molecules into the cells, the next step of the method of the invention is determination of whether a first nucleic acid molecule at a discrete, defined location reduces expression of the reporter gene, relative to expression of the reporter gene in a cell in the absence of the first nucleic acid molecule. Reduction of expression of the reporter gene identifies one of the first nucleic acid molecules at a discrete, defined location as a nucleic acid molecule capable of gene silencing.

In a second aspect, the invention features another method for identifying a nucleic acid molecule capable of gene silencing. In this method, a plurality of first nucleic acid molecules that include candidate knockout reagents or encode knockout reagents are deposited onto a surface in discrete, defined locations. Different first nucleic acid molecules are deposited at different discrete, defined locations. The deposited nucleic acid molecules are then contacted with cells under appropriate conditions for entry of the nucleic acid molecules into the cells. The cells are stably or transiently transfected with a second nucleic acid molecule that includes (i) a promoter; (ii) a reporter gene having a 5′ and/or 3′ untranslated region, the reporter gene operably linked to the promoter for expression in the cell; and (iii) a target nucleic acid derived from the target gene. The target nucleic acid is located within either untranslated region. The nucleic acid molecules are introduced into the cells at the location in which each of the nucleic acid molecules was deposited. Following introduction of the nucleic acid molecules into the cells, the next step of the method of the invention is to determine whether a first nucleic acid molecule at a discrete, defined location reduces expression of the reporter gene, relative to expression of the reporter gene in a cell in the absence of the first nucleic acid molecule. Reduction of expression of the reporter gene identifies the first nucleic acid molecule at a discrete, defined location as a nucleic acid molecule capable of gene silencing.

In a third aspect, the invention features yet another method for identifying a reagent capable of silencing of a target gene. This method includes the steps of: (a) introducing into a cell: a candidate reagent (e.g., a double-stranded RNA molecule or a DNA molecule encoding a double-stranded RNA molecule); and an expression vector that includes (i) a promoter; (ii) a reporter gene having a 5′ and/or 3′ untranslated region, the reporter gene operably linked to the promoter for expression in the cell; and (iii) a target nucleic acid derived from the target gene, the target nucleic acid located within either untranslated region; and (b) determining whether the candidate reagent reduces expression of the reporter gene, relative to expression of the reporter gene in a cell in the absence of the reagent. Reduction of expression of the reporter gene identifies the reagent as a reagent capable of silencing of the target gene.

In a fourth aspect, the invention features another method for identifying a reagent capable of silencing of a target gene. This method includes the steps of: (a) providing: (i) a first cell having a candidate knockout reagent (e.g., a double-stranded RNA molecule or a DNA molecule encoding a double-stranded RNA molecule); and an expression vector having a promoter; a reporter gene comprising a 5′ and/or 3′ untranslated region, the reporter gene operably linked to the promoter for expression in the cell; and a target nucleic acid derived from the target gene, the target nucleic acid located within either untranslated region; and (ii) a second cell having the expression vector but not having the candidate reagent; and (b) determining whether expression of the reporter gene is reduced in the first cell, relative to expression of the reporter gene in the second cell. Reduction of expression of the reporter gene in the first cell identifies the reagent as a reagent capable of silencing of the target gene.

In a fifth aspect, the invention features still another method for identifying a reagent capable of silencing of a target gene. This method includes the steps of: (a) providing (i) a first cell having a candidate knockdown reagent (e.g., a double-stranded RNA molecule or a DNA molecule encoding a double-stranded RNA molecule); and a first expression vector having a promoter; a reporter gene having a 5′ and/or 3′ untranslated region, the reporter gene operably linked to the promoter for expression in the cell; and a target nucleic acid derived from the target gene, the target nucleic acid located within either untranslated region; and (ii) a second cell having the reagent and a second expression vector having the promoter and the reporter gene operably linked to the promoter but not having the target nucleic acid; and (b) determining whether expression of the reporter gene is reduced in the first cell, relative to expression of the reporter gene in the second cell. Reduction of expression of the reporter gene in the first cell identifies the reagent as a reagent capable of silencing of the target gene.

In any of the foregoing aspects, the reporter gene can be any gene that encodes a gene product that can be quantitatively or qualitatively measured or that in turn regulates the expression of another gene that can be quantitatively or qualitatively measured. One exemplary reporter gene encodes green fluorescent protein. Other reporter genes encode chloramphenicol acetyl transferase, luciferase, beta-galactosidase, alkaline phosphatase, beta-lactamase, SALMON-gal, and MAGENTA-gal. Desirably, the promoter is operative in a mammalian cell (e.g., a human, monkey, or mouse cell).

Desirably, the cells employed in the methods of the invention are eukaryotic cells; more desirably, the cells are mammalian cells (e.g., human, monkey, or mouse cells).

The nucleic acid molecules may be components of nucleic acid molecule-containing mixtures, the mixtures also including a carrier such as a gelatin (e.g., a protein gelatin, a hydrogel, a sugar-based gelatin, or a synthetic gelatin) or a nucleic acid stabilizer (e.g., a sugar). When employed, a gelatin is desirably employed at a concentration in the nucleic acid molecule-containing mixture ranging from about 0.01% to about 0.5%, and more desirably at a concentration from about 0.1% to about 0.2%.

The cells are plated onto the surface bearing the transfection array in sufficient density and under appropriate conditions for introduction/entry of the nucleic acid into the cells. Preferably, the cells (in an appropriate medium) are plated on the array at high density (e.g., on the order of 1×10⁵/cm² to 5×10⁵/cm²), in order to increase the likelihood that transfection will occur. For example, the density of cells can be from about 3×10⁴/cm² to about 3×10⁵/cm², and in specific embodiments, is from about 5×10⁴/cm² to about 2×10⁵/cm² and from about 5×10⁴/cm² to about 1×10⁵/cm². The appropriate conditions for introduction/entry of DNA into cells will vary depending on the quantity of cells used.

The nucleic acid molecule-containing mixtures may also include one or more additional components (e.g., a buffer that facilitates nucleic acid molecule condensation or an appropriate transfection reagent).

In one embodiment, the first and/or second nucleic acid molecules are contained in a vector (e.g., an episomal vector or a chromosomally integrated vector). The vector can be, for example, a plasmid or a viral-based vector.

The nucleic acid molecules can be deposited on any suitable surface. Exemplary surfaces that are suitable are glass, polystyrene, and plastic.

Any number of different discrete, defined locations of nucleic acid molecules can be deposited. Desirably, the number of different discrete, defined locations is at least 96, 192, 384, or even 1,000 or 10,000. Each of the discrete, defined locations desirably about 100-200 μm in diameter and about 200-500 μm apart from its nearest adjacent discrete, defined location. Target sequences are desirably arrayed in an addressable fashion, such as rows and columns where the substrate is a planar surface. If each location size is about 100 μm on a side, each chip can have about 10,000 target sequence addresses (locations) in a one centimeter square (cm²) area. In certain preferred embodiments, the transfection array provides a density of at least 10³ different locations per square centimeter (10³ sequences/cm²), and more preferably at least 10⁴ locations/cm², 10⁵ locations/cm², or even at least 10⁶ locations/cm². Of course, lower densities are contemplated, such as at least 100 locations/cm².

In certain embodiments, the transfection array provides multiple different target sequences at each location, e.g., in order to promote co-transfection of the host cells with at least two different target sequences. Co-transfection refers to the simultaneous introduction of two or more plasmids or other nucleic acid constructs into the same cell.

Co-transfections can be performed with transfected cell microarrays if the solution spotted on the surface where reverse transfection occurs contains more than one plasmid or nucleic acid construct. Of course, the collection of different nucleic acid molecules in one location should be distinct from other locations of the array. The co-transfection locations can include, for example, 2-10 different nucleic acid molecules per location, 10-100 different nucleic acid molecules per location, or even more than 100 different nucleic acid molecules per location.

The invention also features a surrogate means for testing the effectiveness of knockout reagents. The expression vector contains a fragment of a target gene in the 5′ or 3′ untranslated region (UTR) of a reporter gene. When this reporter construct is present in a cell, it produces a detectable or assayable reporter gene product. When an effective knockout is present in this same cell, the amount of reporter gene product is reduced.

The assay system allows for the testing of many target nucleic acids with many knockout reagents in a short amount of time. The system can be used for evaluating both the specificity and the relative activity of different knockout reagents. For example, to determine specificity of a knockout reagent of interest, the reagent is introduced into cells individually with a panel of reporter genes, each containing a different target nucleic acid in the 5′ or 3′ UTR of the reporter gene. Relative activity of different knockout reagents is determined by introducing into cells a single reporter plasmid containing the mRNA sequence(s) being targeted individually with each of the knockout reagents under evaluation.

“Protein” or “polypeptide” or “polypeptide fragment” means any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “transformed cell” or “transfected cell” is meant a cell into which (or into an ancestor of which) has been introduced a nucleic acid molecule.

As used herein, “gene” refers to a nucleic acid (e.g., DNA, RNA) sequence that includes coding sequences necessary for the production of a polypeptide. The polypeptide can be encoded by a full length coding sequence or by any portion of the coding sequence. The term “gene” encompasses both cDNA and genomic forms of a gene.

As used herein, the term “gene silencing” refers to a phenomenon whereby gene expression or function is completely or partially inhibited. Throughout the specification, the terms “silencing,” “inhibiting,” “knocking down,” “knocking out” and “suppressing,” when used with reference to gene expression or function, are used interchangeably.

As used herein, the term “oligonucleotide” is defined as a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, in vitro transcription, or a combination thereof.

By “knockout reagent” or “knockdown reagent” is meant a reagent that completely or partially inhibits gene expression or function. Examples of such knockout reagents include dsRNA (e.g., shRNA), siRNA, mRNA-cDNA hybrids (described, e.g., in U.S. Patent Publication No. 2002106686), ribozymes, triple helix forming oligonucleotides (e.g. Majumdar A et al., J. Biol. Chem. (2003) 278:11072-11077), peptide nucleic acids and other modified nucleic acids, DNA-based enzymes, and antisense nucleic acids.

By “target gene” is meant a targeted nucleic acid sequence, the expression of which is desirably silenced. A “target nucleic acid” is a portion of the target gene.

As used herein, the term “vector” refers to a nucleic acid molecule capable of transporting another nucleic acid to that it has been linked. One type of vector is a genomic integrated vector, or “integrated vector,” which can become integrated into the chromosomal DNA of the host cell. Another type of vector is an episomal vector, i.e., a nucleic acid capable of extra-chromosomal replication. Vectors capable of directing the expression of genes to that they are operatively linked are referred to herein as “expression vectors.” In the present specification, “plasmid” and “vector” are used interchangeably unless otherwise clear from the context.

The term “loss-of-function,” as it refers to genes inhibited by a knockout reagent, refers a diminishment in the level of expression of a gene when compared to the level in the absence of the knockout reagent.

The term “expression,” with respect to a gene sequence, refers to transcription of the gene and, as appropriate, translation of the resulting mRNA transcript to a protein. Thus, as will be clear from the context, expression of a protein coding sequence results from transcription and translation of the coding sequence.

“Transient transfection” refers to cases where exogenous DNA does not integrate into the genome of a transfected cell, e.g., where episomal DNA is transcribed into mRNA and translated into protein.

The term “location,” as it is used in describing a transfection array, refers to an area of a substrate having a homogenous collection of a target sequence (or sequences in the case of certain co-transfection embodiments). One location is different from another location if the target sequences of the different location have different nucleic acid molecules.

A cell has been “stably transfected” with a nucleic acid construct when the nucleic acid construct is capable of being inherited by daughter cells. This state is generally typified by the integration of the transfected DNA into the host cells genome.

As used herein, a “reporter gene construct” is a nucleic acid that includes a “reporter gene” operatively linked to at least one transcriptional regulatory sequence. Transcription of the reporter gene is controlled by these sequences to which they are linked.

By “transformation” or “transfection” is meant any method for introducing foreign molecules, for example, an antisense nucleic acid, into a cell. Lipofection, calcium phosphate precipitation, retroviral delivery, electroporation, biolistic transformation, and penetratin are just a few of the methods that may be used.

By “antisense” is meant a nucleic acid sequence, regardless of length, that is complementary to the coding strand or mRNA of a target gene.

By “ribozyme” is meant an RNA that has enzymatic activity, possessing site specificity and cleavage capability for a target RNA molecule. Ribozymes can be used to decrease expression of a polypeptide. Methods for using ribozymes to decrease polypeptide expression are described, for example, by Turner et al. (2000) Adv. Exp. Med. Biol. 465:303-318 and Norris et al. (2000) Adv. Exp. Med. Biol. 465:293-301.

By “positioned for expression” is meant that the DNA molecule is positioned adjacent to a DNA sequence that directs transcription and translation of the sequence (i.e., facilitates the production of a recombinant protein or an RNA molecule).

By “reporter gene” is meant a gene whose expression may be directly or indirectly assayed; such genes include, without limitation, green fluorescent protein (GFP), beta-glucuronidase (GUS), luciferase, chloramphenicol transacetylase (CAT), beta-galactosidase, beta-lactamase, red fluorescent protein, alkaline phosphate, and horseradish peroxidase. An example of a reporter gene whose expression may be indirectly measured is one encoding a transcription factor, which, in turn, drives expression of a second gene. Measurement of expression of that second gene indirectly measures expression of the gene encoding the transcription factor.

By “promoter” is meant a minimal sequence sufficient to direct transcription. Also included in the invention are those promoter elements that are sufficient to render promoter-dependent gene expression controllable for cell type-specific, tissue-specific or that are inducible by external signals or agents; such elements may be located in the 5′ or 3′ regions of the native gene.

By “operably linked” is meant that a gene and one or more regulatory sequences are connected in such a way as to permit gene expression when the appropriate molecules (e.g., transcriptional activator proteins) are bound to the regulatory sequences.

Using the assay system described here, a single reporter construct is generated for each protein whose knockdown is desired. mRNA-targeted knockdown reagents for any one protein of interest can be evaluated using this single reporter construct. The function of this assay system is independent of the nature of the reporter readout (as described below). The only requirement for the reporter is that the specific sequence toward which the knockdown reagent is targeted is contained within the fragment inserted into the 5′ or 3′ UTR of the reporter gene. This assay system increases the efficiency by which knockout reagents are evaluated for both specificity and relative activity.

Other features and advantages of the invention will be apparent from the following description of the preferred embodiments thereof and from the claims.

### BRIEF DESCRIPTION OF THE DRAWINGS

FIGS. 1A and 1B are schematic illustrations of reporter assay vector components. A generic assay system vector is shown in FIG. 1A. The sequences for conferring resistance in bacterial cells are present so that the vector can be produced in bacteria in large quantities, but are not necessary for the operation of the reporter assay vector of the invention. The specific vector used for this study is shown in FIG. 1B.

FIG. 2 is a schematic illustration showing the results following forward co-transfection of reporter plasmids with knockout plasmids. Hek293T cells were forward co-transfected with reporter plasmids and knockout plasmids. The four reporter plasmids are each present at 20 ng/μl and are present in each row of wells as noted on the left. The knockout plasmids are shown in columns and range from 0 ng/μl (left most column) to a mass excess of 6.5× (130 ng/μl; columns 4 and 5). Each photograph represents a single well in a 96-well plate and all images were captured with identical exposure settings.

FIG. 3 summarizes the DNA sequences introduced into the parent construct. The XhoI loop is underlined in each case. The p53 KO and dnmt KO sequences were cloned into the BamHI-HindIII sites, while all of the cdk2 knockout sequences were cloned into the AleI-EcoRI sites.

FIG. 4 is a schematic illustration showing the results of a surrogate screening assay to identify functional knockdown reagents targeting the cyclin-dependant kinase 2 (cdk2) sequence. Four different plasmids targeting four different regions of the gene were created as described in FIG. 3. Each was forward-transfected against three different reporter constructs at a mass ratio of 6.5 to 1. The three reporter constructs are pd1-EGFP, d1EGFP-p53 (which contains approximately 1 kb of p53 sequence in the 3′UTR), and d1EGFP-cdk2. d1EGFP-cdk2 was created using the following oligonucleotide sequences: 5′-GAATTGGCTAGGCGCGGCCGCTCACATCCTGG AAGAAAGGG-3′ (SEQ ID NO: 1) and 5′-GGCGACGTCGGAGCGGCCGCGAAT TCAGCCAGAAACAAGTTGACGG-3′ (SEQ ID NO: 2) to amplify and clone a 0.9 kb fragment of cdk2 sequence into the NotI site in the 3′ UTR of d1-EGFP. Transfected cells were photographed at 48 hours, using identical camera settings. Each picture is a single well of a 96-well plate.

FIGS. 5A and 5B are schematic illustrations showing that plasmid-based p53 siRNA reduces expression of p53-EGFP fusion protein and of d1EGFP with a 3′UTR containing the p53 knockout target. HEK293T cells were reverse transfected with either a p53-EGFP fusion protein reporter (A) or a d1EGFP reporter with or without a p53 knockout target sequence in its 3′UTR (B). The indicated ratio of knockout target plasmid to reporter plasmid was used. Forty-eight hours post-transfection, GFP fluorescence was quantified. Displayed is the average level of GFP fluorescence quantified from a minimum of 192 images per transfection condition.

### DETAILED DESCRIPTION OF THE INVENTION

We have discovered methods for the rapid identification of knockdown reagents directed against a specific target gene. Using this method, described below, one can assay any of a number of candidate knockout reagents for activity against the target nucleic acid.

The methods of the invention are described using reagents that operate through RNAi (e.g., dsRNA, shRNA), but one skilled in the art will recognize that the methods are equally applicable for screening other types of knockout reagents (e.g., antisense RNA, ribozymes, mRNA-cDNA hybrids, triple helix forming oligonucleotides). In one form of RNAi used in the method described here, a plasmid-based expression system is used to transcribe an RNA sequence that is predicted to form a hairpin structure in the cell. These small hairpin RNAs (shRNAs) are processed into siRNAs within the cell. The siRNAs are targeted to specific endogenous mRNAs in a homology-dependent manner. The homology dependent pairing of siRNAs to an endogenous mRNA targets the endogenous mRNA for degradation.

To utilize the assay system of the present invention, the DNA sequence encoding the mRNA sequence that is to be targeted by the knockout reagent(s) is cloned into the 5′ or 3′ UTR of the reporter gene, using, for example, the above described restriction site or MCS to create a reporter construct. This reporter construct is then transfected into appropriate host cells (e.g., mammalian cells, yeast cells, insect cells, Drosophila cells), along with a reagent designed to knockout the sequence of interest. The two transfections can be contemporaneous (i.e., co-transfections) or temporally separated. Any means for transfection is suitable. Knockout reagents capable of interfering with the target mRNA are detected by a reduction of signal in the reporter system.

The screening assay of the invention may be performed in an array format to screen tens, hundreds, or even thousands of candidate knockout reagents simultaneously. In one particularly desirable embodiment, the assay system of the present invention is used in conjunction with the high throughput capabilities of the reverse transfection system, described herein. This format allows the performance of multiple co-transfections at a given time and therefore to screen hundreds or thousands of different reagents rapidly.

Transfection

The nucleic acids used in the transfection arrays of the present invention can be, for example, DNA, RNA or modified or hybrid forms thereof. The nucleic acids may be from any of a variety of sources, such as nucleic acid isolated from cells, or that which is recombinantly produced or chemically synthesized. All or a portion of the nucleic acid sequences can be synthesized chemically. In such a manner, random and semi-random sequence can be introduced into the target sequences, as well as modified forms of nucleotides and nucleotide linkages, such as the use of modified backbones, methylated nucleotides and the like.

In general, it will be desirable that the reporter construct be capable of replication in the host cell. The reporter construct may be a DNA that is integrated into the host genome, and thereafter is replicated as a part of the chromosomal DNA, or it may be DNA that replicates autonomously, as in the case of an episomal plasmid. In the latter case, the vector will include an origin of replication that is functional in the host. In the case of an integrating vector, the vector may include sequences that facilitate integration, e.g., sequences homologous to host sequences, or encoding integrases. The use of retroviral long terminal repeats (LTR) or adenoviral inverted terminal repeats (ITR) in the construct of the transfection array can, for example, facilitate the chromosomal integration of the construct.

Appropriate cloning and expression vectors for use with bacterial, fungal, yeast, and mammalian cellular hosts are known in the art, and are described in, for example, Powels et al. (Cloning Vectors: A Laboratory Manual, Elsevier, New York, 1985). Such vectors may be readily adapted for use in the present invention. The expression vectors may comprise non-transcribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

Certain preferred mammalian expression vectors contain both prokaryotic sequences, to facilitate the propagation of the vector in bacteria (such as in an amplification step after recovery from the array), and one or more eukaryotic transcription units for expressing the target sequence in eukaryotic host cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors which can be readily adapted for use in the subject method. Some of these vectors are modified with sequences from bacterial plasmids, such as pBR322, to facilitate replication and drug resistance selection in both prokaryotic and eukaryotic cells. Alternatively, derivatives of viruses, such as the bovine papillomavirus (BPV-1), or Epstein-Barr virus (pHEBo, pREP-derived and p205) and the like, can be used to derive the subject arrays. The various methods employed in the preparation of the plasmids are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press: 1989), Chapters 16 and 17.

Particularly desirable vectors contain regulatory elements that can be linked to the reporter construct for expression in mammalian cells, and include are cytomegalovirus (CMV) promoter-based vectors such as pcDNA1 (Invitrogen, San Diego, Calif.), MMTV promoter-based vectors such as pMAMNeo (Clontech, Palo Alto, Calif.) and pMSG (Pharmacia, Piscataway, N.J.), and SV40 promoter-based vectors such as pSVO (Clontech, Palo Alto, Calif.).

Co-transfections can be performed such that more than one plasmid or nucleic acid construct is introduced during a single transfection. The co-transfection can include, for example, 2-10 different nucleic acid molecules, 2-100 different nucleic acid molecules, or even more than 100 different nucleic acid molecules. Typically, one of the nucleic acids transfected in a co-transfection is a reporter construct of the invention.

Examples of reporter genes include, but are not limited to CAT (chloramphenicol acetyl transferase; Alton and Vapnek (1979) Nature 282:864-869) luciferase and other enzyme detection systems, such as beta-galactosidase; firefly luciferase (deWet et al. (1987) Mol. Cell. Biol. 7:725-737); bacterial luciferase (Engebrecht and Silverman (1984) PNAS 1:4154-4158; Baldwin et al. (1984) Biochemistry 23:3663-3667); alkaline phosphatase (Toh et al. (1989) Eur. J. Biochem. 182:231-238; Hall et al. (1983) J. Mol. Appl. Gen. 2:101), human placental secreted alkaline phosphatase (Cullen and Malim (1992) Methods Enzymol. 216:362-368); beta-lactamase, glutathione-S-transferase, C₁₂FDG, SALMON-gal (6-Chloro-3-indoxyl-beta-D-galactopyranoside) (Biosynth AG, Staad, Switzerland), MAGENTA-Gal (5-Bromo-6-chloro-3-indoxyl-beta-D-galactopyranoside), (Biosynth AG), horseradish peroxidase, exo-glucanase (product of yeast exbl gene; nonessential, secreted), and green fluorescent protein (GFP). The reporter gene may also be selectable, creating, for example, a difference in the growth or survival rate between cells that express the reporter gene and those that do not.

Double-Stranded RNA

In one embodiment of the invention, the candidate knockout reagents include double-stranded RNA (dsRNA) molecules. Typically, dsRNAs are about 21 or 22 base pairs, but may be shorter or longer if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. (2002) Science 296:550-553; Paddison et al. (2002) Genes & Devel. 16:948-958; Paul et al. (2002) Nature Biotechnol. 20:505-508; Sui et al. (2002) Proc. Natl. Acad. Sci. USA 99:5515-5520; Yu et al. (2002) Proc. Natl. Acad. Sci. USA 99:6047-6052; Miyagishi et al. (2002) Nature Biotechnol. 20:497-500; and Lee et al. (2002) Nature Biotechnol. 20:500-505, each of which is hereby incorporated by reference.

Libraries of randomized or semi-randomized sequences are constructed, e.g., as a collection of shRNA or siRNA encoding plasmids by randomizing the nucleotide sequence of the stem portion of the molecule. This is accomplished by synthetic means (e.g., oligo synthesis) or non-synthethic means (e.g., subcloning small fragments of cDNA or genomic DNA). Individual clones are isolated using standard molecular biological techniques (e.g., colony isolation) and reverse transfected against a series of reporter plasmids. Clones that affect reporter gene expression are then further characterized.

Small Hairpin RNA

Small hairpin RNAs consist of a stem-loop structure with optional 3′ UU-overhangs. While there may be variation, stems can range from 21 to 31 bp (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

Reverse Transfection

The screening methods of the invention desirably are performed using reverse transfection. In reverse transfection, nucleic acid molecules are affixed to a surface, which is then contacted with cells to be transfected under conditions appropriate for entry of the nucleic acid molecules into the cells at the location of each contacting. This method allows for the generation of arrays of transfected cells, e.g., on a single slide or other surface.

In one embodiment of the method, one or more nucleic acid molecule-containing mixtures (each including desired nucleic acid molecules and, optionally, a carrier or a nucleic acid stabilizer) are deposited onto a surface, such as a slide, in discrete, defined locations. The mixture is allowed to affix to the slide. For example, a nucleic acid-containing mixture can be spotted onto a slide, such as a glass slide coated with Σ poly-L-lysine, for example, by hand or using a microarrayer. The mixtures can be affixed to the slide by, for example, subjecting the resulting product to drying at room temperature, at elevated temperatures or in vacuum-desiccators. The length of time desirable to affix the nucleic acid molecule-containing mixture depends on several factors, such as the quantity of mixture placed on the surface, and the temperature and humidity conditions used.

The concentration of nucleic acid molecules present in any mixture can be determined empirically, but will generally be in the range of from about 0.01 μg/μl to about 0.2 μg/μl and, in specific embodiments, is from about 0.02 μg/μl to about 0.10 μg/μl. Alternatively, the concentration of nucleic acid molecules present in a mixture can be from about 0.01 μg/μl to about 0.5 μg/μl, from about 0.01 μg/μl to about 0.4 μg/μl and from about 0.01 μg/μl to about 0.3 μg/μl. Similarly, the concentration of gelatin or other carrier can be determined empirically for each use, but will generally be in the range of 0.01% to 0.5% and, in specific embodiments, is from about 0.05% to about 0.5%, from about 0.05% to about 0.2% or from about 0.1% to about 0.2%. If the nucleic acid molecules are present in a vector, the vector can be of any type, such as a plasmid or viral-based vector, into which the nucleic acid molecules can be introduced and expressed in recipient cells. For example, a CMV-driven expression vector can be used. Commercially available plasmid-based vectors or viral-based vectors can be used.

In certain embodiments, the transfection array provides, in a single array, e.g., preferably at least 10 different sequences, more preferably at least 100, 1000 or even 10,000 different, discrete sequences. Target sequences are typically arrayed in an addressable fashion, such as rows and columns where the substrate is a planar surface. If each location size is about 100 microns on a side, each chip can have about 10,000 target sequence addresses (locations) in a one centimeter square (cm²) area. In certain preferred embodiments, the transfection array provides a density of at least 10³ different locations per square centimeter (10³ sequences/cm²), and more preferably at least 10⁴ locations/cm², 10⁵ locations/cm², or even at least 10⁶ locations/cm². Of course, lower densities are contemplated, such as at least 100 locations/cm² or fewer.

In certain embodiments, the transfection array provides multiple different candidate reagent at each location, e.g., in order to promote co-transfection of the host cells with at least two different target sequences. Co-transfection refers to the simultaneous introduction of two or more nucleic acid molecules into the same cell. The co-transfection locations can include, for example, 2-10 different candidate reagents per location, 10-100 different candidate reagents per location, or even more than 100 different candidate reagents per location.

The carrier for use in the methods of the present invention can be, for example, gelatin or a nucleic acid stabilizer (e.g., a sugar). In certain embodiments, the carrier is a hydrogel, such as polycarboxylic acid, cellulosic polymer, polyvinylpyrrolidone, maleic anhydride polymer, polyamide, polyvinyl alcohol, or polyethylene oxide.

Any suitable surface, which can be used to affix the nucleic acid containing mixture to its surface, can be used. For example, the surface can be glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose), paper, biomaterials (such as protein, gelatin, agar), tissues (such as skin, endothelial tissue, bone, cartilage), minerals (such as hydroxyapatite, graphite). Additional compounds may be added to the base material of the surface to provide functionality. For example, scintillants can be added to a polystyrene substrate to allow scintillation proximity assays to be performed. The substrate may be a porous solid support or non-porous solid support. The surface can have concave or convex regions, patterns of hydrophobic or hydrophilic regions, diffraction gratings, channels or other features. The scale of these features can range from the meter to the nanometer scale. For example, the scale can be on the micron scale for microfluidics channels or other MEMS features or on the nanometer scale for nanotubes or buckyballs. The surface can be planar, planar with raised or sunken features, spherical (e.g. optically encoded beads), fibers (e.g. fiber optic bundles), tubular (both interior or exterior), a 3-dimensional network (such as interlinking rods, tubes, spheres) or other shapes. The surface can be part of an integrated system. For instance, the surface can be the bottom of a microtitre dish, a culture dish, a culture chamber. Other components, such as lenses, gratings, and electrodes, can be integrated with the surface. In general, the material of the substrate and geometry of the array will be selected based on criteria that it be useful for automation of array formation, culturing and/or detection of cellular phenotype.

In still other embodiments, the solid support is a microsphere (bead), especially a FACS sortable bead. Preferably, each bead is an individual location, e.g., having a homogenous population of target sequences and distinct from most other beads in the mixture, and one or more tags which can be used to the identify any given bead and therefore the target sequence it displays. The identity of any given target sequence that can induce a FACS-detectable change in cells that adhere to the beads can be readily determined from the tag(s) associate with the bead. For example, the tag can be an electrophoric tagging molecules that are used as a binary code (Ohlmeyer et al. (1993) PNAS 90:10922-10926). Exemplary tags are haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 2⁴⁰ (e.g., upwards of 10¹²) different molecules. A more versatile system has, however, been developed that permits encoding of essentially any combinatorial library. Here, the compound would be attached to the solid support via the photocleavable linker and the tag is attached through a catechol ether linker via carbene insertion into the bead matrix (Nestler et al. (1994) J. Org. Chem. 59:4723-4724). This orthogonal attachment strategy permits the FACS sorting of the cell/bead entities and subsequent decoding by ECGC after oxidative detachment of the tag sets from isolated beads. In other embodiments, the beads can be tagged with two or more fluorescently active molecules, and the identity of the bead is defined by the ratio of the various fluorophores.

In still another embodiment, the transfection array can be disposed on the end of a fiber optic system, such as a fiber optic bundle. Each fiber optic bundle contains thousands to millions of individual fibers depending on the diameter of the bundle. Changes in the phenotype of cells applied to the transfection array can be detected spectrophotometrically by conductance or transmittance of light over the spatially defined optic bundle. An optical fiber is a clad plastic or glass tube wherein the cladding is of a lower index of refraction than the core of the tube. When a plurality of such tubes are combined, a fiber optic bundle is produced. The choice of materials for the fiber optic will depend at least in part on the wavelengths at which the spectrometric analysis of the transfected cells is to be accomplished.

In addition, the surface can be coated with, for example, a cationic moiety. The cationic moiety can be any positively charged species capable of electrostatically binding to negatively charged polynucleotides. Preferred cationic moieties for use in the carrier are polycations, such as polylysine (e.g., poly-L-lysine), polyarginine, polyomithine, spermine, basic proteins such as histones (Chen et al. (1994) FEBS Letters 338:167-169), avidin, protamines (see e.g., Wagner et al. (1990) PNAS 87: 3410-3414), modified albumin (i.e., N-acylurea albumin) (see e.g., Huckett et al. (1990) Chemical Pharmacology 40: 253-263), and polyamidoamine cascade polymers (see e.g., Haensler et al. (1993) Bioconjugate Chem. 4:372-379). A preferred polycation is polylysine (e.g., ranging from 3,800 to 60,000 daltons). Alternatively, the surface itself can be positively charged (such as gamma amino propyl silane or other alkyl silanes). The surface can also be coated with molecules for additional functions. For instance, these molecules can be capture reagents such as antibodies, biotin, avidin, Ni-NTA to bind epitopes, avidin, biotinylated molecules, or 6-His tagged molecules. Alternatively, the molecules can be culture reagents such as extracellular matrix, fetal calf serum, or collagen.

### EXAMPLE

DNA Construction

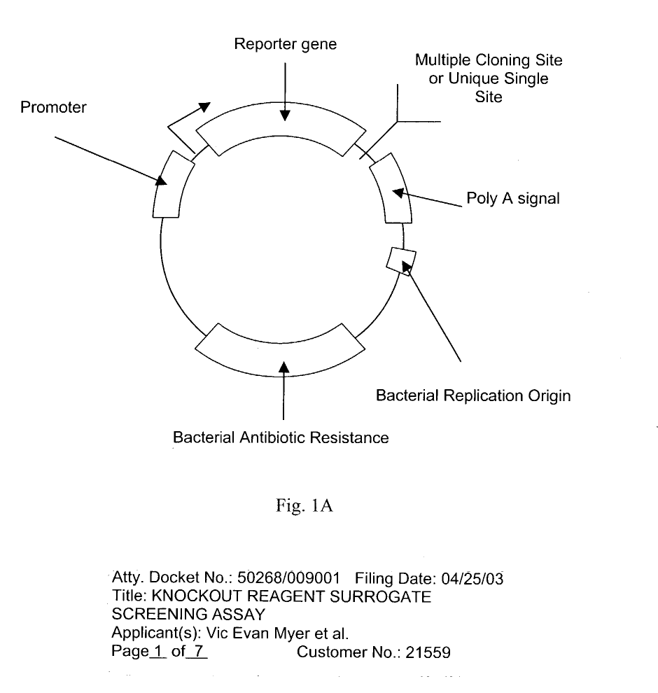

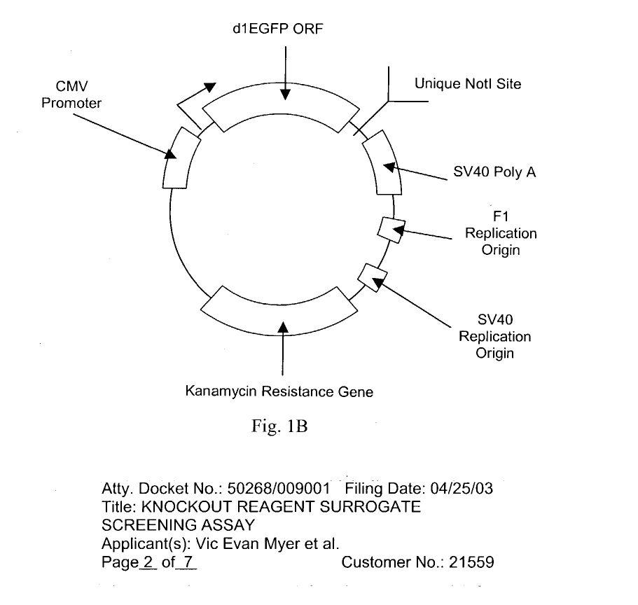

As described herein, the components of the reporter construct used in the assay system of the present invention are (i) a promoter that is active in mammalian cells, (ii) a reporter gene, (iii) a multiple cloning site or unique single site in the 3′UTR, and (iv) a polyadenylation signal. A schematic illustration of such a construct is shown in FIG. 1A. In the experiments described herein, we selected the parent construct pd1EGFP-N1. This construct has a CMV promoter driving the expression of a destabilized version of green fluorescent protein (GFP). This protein is assayable either by direct examination of the cells for fluorescence or by immunochemical means. The construct contains a single NotI site in its 3′ UTR and concludes with a SV40 polyadenylation site. The resulting construct is shown in FIG. 1B. We designed the two PCR primers, p53UTR1 (5′-GGCGACGTCGGAGCGGCCGCGAATTCGG ATGATTTGATGCTGTCCC-3′; SEQ ID NO: 3)) and p53UTR2 (5′-GAATTGGCT AGGCGCGGCCGCCTTTTTGGACTTCAGGTGGC-3′; SEQ ID NO: 4), to amplify a portion of the p53 ORF and clone it into the NotI site of pd1EGFP-N1. The construct created (pd1EGFP-p53) contains approximately 1 kb of DNA sequence derived from the p53 ORF in its 3′ UTR.

Forward Transfection

Design

To test the ability of knockout reagents to affect the levels of reporter expression we first performed transfections in 96-well plates using conventional transfection protocols. The four reporter constructs used for the experiment were pEGFP-N1 (BD-Clontech catalog # 6085-1), which encodes a codon optimized version of GFP, p53-EGFP (BD-Clontech catalog #6920-1) which encodes a fusion protein composed of p53 on the N-terminal end and GFP on the C-terminal end, pd1EGFP-N1 (BD-Clontech catalog #6073-1) which encodes a destabilized version of GFP, and pd1EGFP-p53 (the reporter construct described above). Effective knockout reagents directed towards p53 would be expected to reduce fluorescence of the two reporters containing p53 sequence (p53-EGFP and pd1EGFP-p53) and not affect the others.

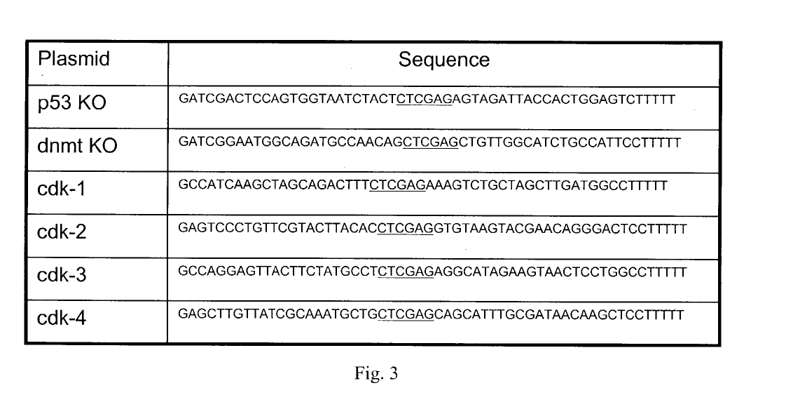

For knockout reagents we selected two plasmids, each encoding a previously identified shRNA (Brummelkamp, et al.; Sui, et al.). p53 KO is directed against p53 and dnmt1 KO is directed against dnmt1; the latter serves as a negative control for this experiment. The sequences are shown in FIG. 3.

All transfections contained a total of 150 ng of DNA and the amount of reporter construct was 20 ng in all wells. The knockout plasmids were added at increasing amounts of 20 ng (1:1), 75 ng (3.75:1) and 130 ng (6.5:1). To bring the total amount of DNA to 150 ng, pBluescript was added when necessary. Transfections were performed using Superfect™ (Invitrogen, Carlsbad, Calif.) according to the manufacturers directions.

Forty-eight hours post transfection, wells were imaged with a 400 ms exposure on an inverted fluorescence microscope with the appropriate filters to detect GFP.

Results

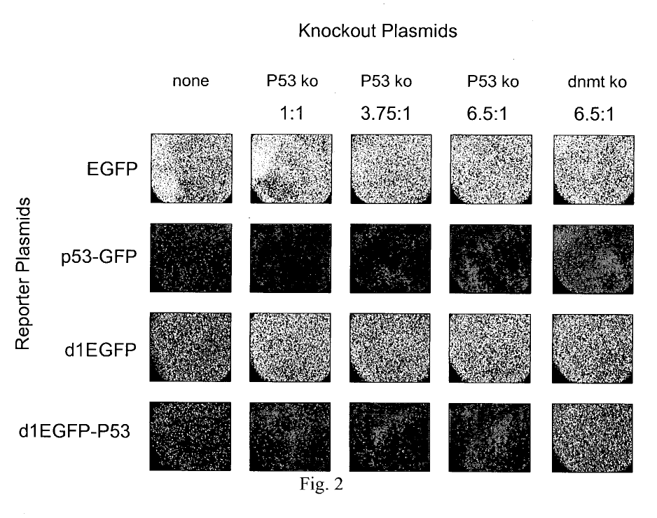

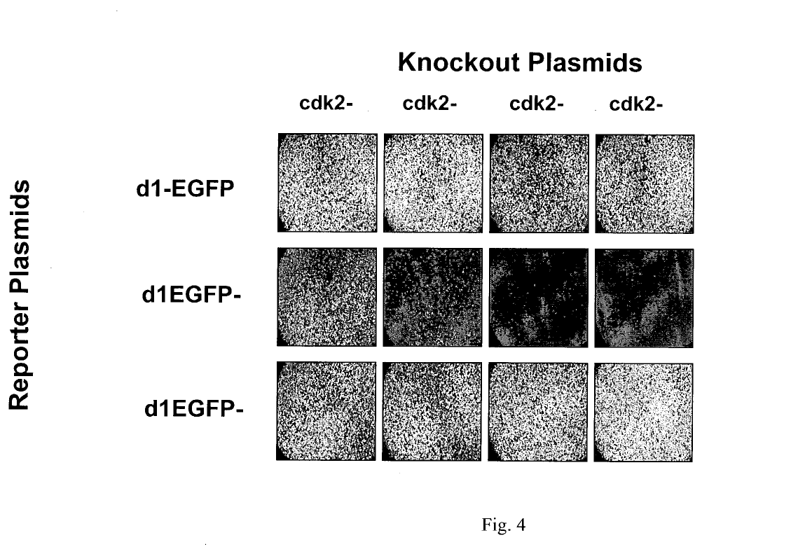

The results are shown in FIGS. 2, 4A, and 4B. The p53 knockout data are shown in FIG. 2. Only the maximum amount of negative control reagent (dnmt1 KO) is shown. As expected, presence of the knockout reagents does not effect the expression of either EGFP or d1EGFP expression. In contrast, co-transfection of the p53 KO plasmid (columns 2-4) with either p53-GFP or d1EGFP-p53 (rows 2 and 4) diminished fluorescent signal. The negative control knockout reagent (dnmt1 KO, column 5) and no knockout reagent (column 1) serve as controls. From these experiments we can conclude that the reporter system is successful in discriminating effective knockout reagents from ineffective ones.

Shown in FIGS. 4A and 4B is the utilization of the surrogate screening assay to identify functional knockdown reagents targeting the cyclin-dependant kinase 2 (cdk2) sequence. Four different plasmids targeting four different regions of the gene were created as described above. Each was forward transfected against three different reporter constructs at a mass ratio of 6.5 to 1. The three reporter constructs are pd1-EGFP, d1EGFP-p53 (which contains approximately 1 kb of p53 sequence in the 3′ UTR) and d1EGFP-cdk2. As can be seen from the figure, the four knockout reagents appear to have specificity for the d1EGFP-cdk2 reporter construct (row 2), but knock down expression at different levels. Clone cdk2-1 has little effect on the reporter plasmid, while cdk2-3 and cdk2-4 almost entirely eliminate fluorescent signal. Cdk2-2 appears to have intermediate inhibitory effect.

Reverse Transfection

Design/Methods

The ability of the knockout reagents to inhibit the expression of a reporter containing the knockout target in the 3′ UTR of the reporter mRNA was evaluated by reverse transfection. The desired plasmid combinations were put into a gelatin solution (gelatin concentration=0.2%; DNA concentration=180 ng/μl). Approximately 1 nl of these DNA/gelatin solutions were robotically spotted in an 8×8 array on the bottom surface of a 96 well tissue culture plate using a PROSYS 5510A printer (Cartesian Technologies, Irvine, Calif.) (spot size=˜120 μm in diameter, with a 350 μm center to center distance). The solution used to print each spot of the array contained 40 ng/μl of a GFP reporter construct. KO plasmids were added to the reporter construct solution in increasing amounts: 1.875 ng/μl, 7.5 ng/μl, 30 ng/μl and 120 ng/μl to achieve different KO:reporter plasmid ratios. The total DNA concentration of each gelatin solution was held constant at 160 ng/μl using pBluescript II KS(+) (Stratagene, La Jolla, Calif.). The dried DNA arrays were incubated with Effectene™ transfection reagent (˜60 uls per well) (Qiagen, Valencia, Calif.). After removal of the transfection reagent (30 minutes), a suspension of HEK293T cells in growth medium was added to each well. The cells form a monolayer on the bottom surface of each well. Cells that adhere to the DNA spots on the well surface are transfected by the DNA, whereas all other parts of the monolayer that do not come into physical contact with spotted DNA remain untransfected and serve as negative controls.

Forty eight hours after adding cells to the DNA array, an inverted fluorescence microscope (Axiovert 200M, Zeiss, Thornwood, N.Y.) with the appropriate GFP detection filters (470 nm excitation/525 nm emission) and a 2.5× objective was used to capture digital images of each well using an 800 ms exposure time. The location of the transfected cells was identified by the presence of GFP fluorescence. Quantification of GFP fluorescence in each spot of transfected cells was obtained from the digital images using ArrayVision™ software (Amersham Bioscences, Piscataway, N.J.).

One method for carrying out the reverse transfection method is described below.

Starting Materials

HEK293T (3.5×10⁷ cells plated in a T-175 flask ˜24 hours prior to transfection)

HEK293T growth media pre-warmed at 37° C.

DMEM High Glucose (Life Technologies 11965-092)

Trypsin-EDTA (Life Technologies 25300-054)

50 ml tubes (VWR 21008-178)

Hemacytometer (VWR Counting chamber 15170-208)

Inverted TC Microscope

Trypan Blue Stain 0.4%—(Life Technologies 15250-061)

Effectene Transfection reagent kit, (Qiagen 301425)

Printed arrays

15 ml tubes (VWR 20171-024)

1.5 ml tubes (VWR 05-402-25)

Multichannel pipette (Finnpipette, VWR 53515-026)

Multichannel reservoir (VWR 21007-972)

Multichannel aspirator (VWR 29443-120 and 29443-002)

Vortex Genie

Protocol

1) Identify array plates to be transfected, remove from storage and equilibrate in TC hood for 15 minutes prior to transfection.

2) Place the cell media in the 37° C. water bath.

3) Prepare the transfection reagent.

4) Expose array to transfection reagent.

a) Deliver 60 μl of reagent to the bottom of each well using a multichannel pipette (Finnpipette, VWR 53515-026).

b) After addition of the reagent, place lid on the plate, and gently rock the plate to ensure complete coverage of the reagent over the surface of the well.

c) Repeat steps (a) and (b) for all plates to be transfected.

d) Incubate arrays for 30 minutes. It is desirable that no single array should be exposed to transfection reagent for more than forty minutes.

5) Harvest and dilute cells.

a) Trypsinize cells.

i) Using a 25 ml pipette remove growth media from T-175 flask(s) containing cells that were prepared for transfection the day prior.

ii) Gently wash cells with 4 ml of 4° C. trypsin-EDTA, adding trypsin to side of flask, not directly onto cells. Coat cell surface and remove trypsin immediately.

iii) Add 2 ml trypsin to flask, evenly distribute over cells, and place in 37° C. incubator for 3-5 minutes for cells to release from the surface.

iv) After cells have trypsinized for 3-5 minutes, remove from the incubator and tip the plate from side to side to release the cells from the flask

v) Add 18 ml of media to resuspend cells and inactivate the trypsin.

vi) Pipette cells up and down ˜10 times with a 10 ml strip pipette to get a single cell suspension, while avoiding frothing of media.

vii) Transfer the cell suspension to a sterile 50 ml conical tube.

b) Count cells.

i) Using a P200 pipette, transfer 100 μl of cell suspension to a 1.5 ml eppendorf tube.

ii) Add 100 μl of Trypan blue stain 0.4% (Invitrogen 15250-061) and mix by pipetting up and down several times. Trypan blue aliquot is stored in 50 ml labeled conical next to the microscope.

iii) Gently pipette a portion of the cell/trypan mixture into the hemacytometer reservoir until the etched region is evenly coated.

iv) Using the microscope with the 10× objective, count the number of live (bright colored, excluding the blue stain) and dead cells (dark colored) in two of the large quadrants containing 16 sub-quadrants.

c) Calculate the cell dilution.

d) Make dilution

i) Set up the required number of 50 ml conical tubes in racks.

ii) Add the cell suspension to each tube.

iii) Add the cell media to each tube.

iv) Mix by inverting the tubes several times.

v) Store cells in 37 C transfection incubator until ready for use.

6) Remove transfection reagent after 30 minutes and wash array wells.

a) Remove transfection reagent from array by aspiration using the 8 channel aspirator (VWR 29443-120 and 29443-002).

b) Repeat the removal of transfection reagents for all plates.

c) Pour serum free DMEM into a sterile multi-channel reservoir (VWR 21007-972).

d) Using a multi-channel pipette, add 100 μl of serum free DMEM to each transfected well.

e) Remove wash media using the using the 8 channel aspirator.

7) Add cells to array wells.

a) Remove cells from incubator and invert tube several times to mix cells thoroughly to ensure even dispersion of cells in solution.

b) Pour cells into a sterile multi-channel reservoir (VWR 21007-972). Maximum volume held by the reservoir is 50 ml.

c) Using a multi-channel pipette (Finnpipette, VWR 53515-026), dispense 100 μl of cells into the bottom of each well at the six o'clock position.

i) Tilt the array plate so that the top of the plate is off the surface of the hood by approximately 2 cm.

ii) Load 12 tips onto the multi-channel pipette set at 100 μl and fill the 12 channel pipette with cell suspension.

iii) Place pipette tips at the 6 o'clock position of the well where the wall and bottom of the well meet.

iv) Dispense the cell solution slowly into wells.

v) Repeat for all wells in a plate.

d) Place lid on array plate and repeat for all array plates.

e) Store array plates in the 37° C. TC incubator designated for transfections.

Results/Summary

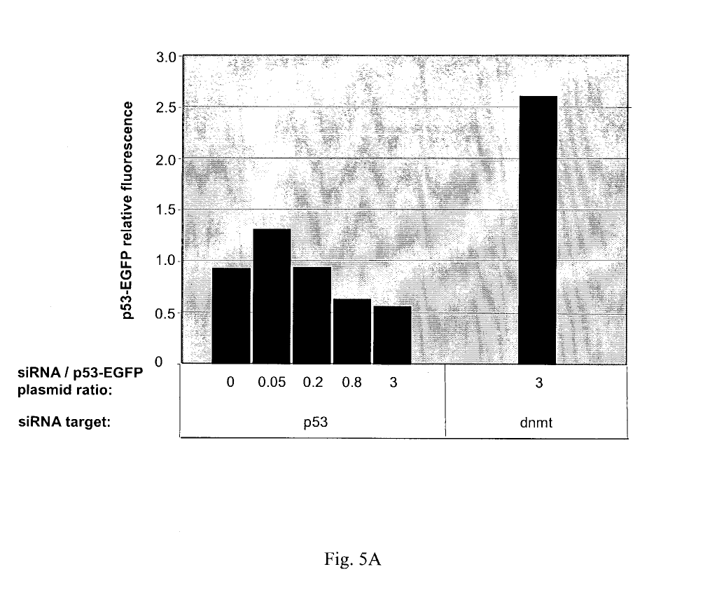

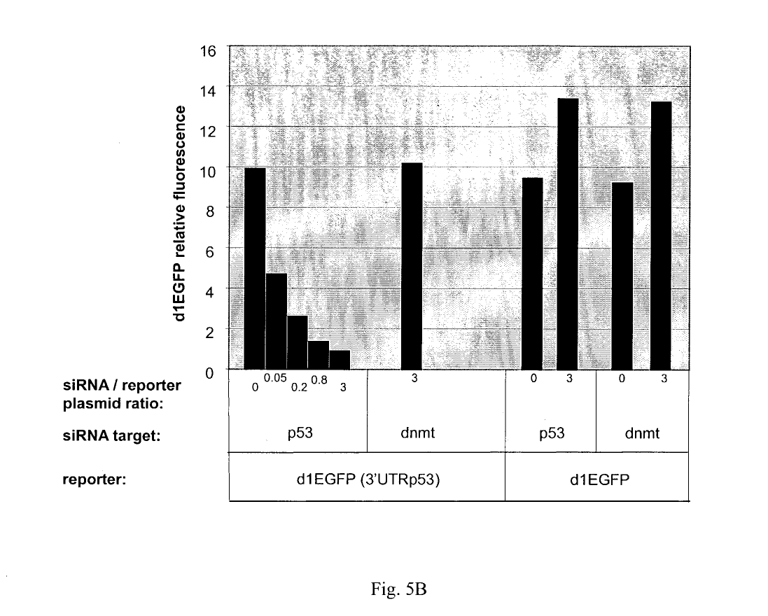

Results are summarized in FIGS. 5A and 5B. FIG. 5A demonstrates that p53-EGFP fusion protein expression, as measured by GFP fluorescence, declines with increasing concentration of the p53 KO plasmid. As expected, GFP fluorescence does not decrease in the presence of the maximum concentration of a dnmt1 KO plasmid. FIG. 5B shows the effect of the p53 and the dnmt1 KO plasmids on a d1EGFP reporter with or without the p53 mRNA knockout target in its 3′UTR. Neither the p53 nor the dnmt1 KO plasmid results in a decline in GFP fluorescence when the d1EGFP plasmid is the reporter. When the d1EGFP reporter containing the p53 mRNA knockout target in its 3′UTR is used, reverse co-transfection of increasing concentrations of p53 KO plasmid, but not dnmt1 KO plasmid, result in decreased levels of GFP fluorescence.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

What we claim is:

1. A method for identifying a nucleic acid molecule capable of gene silencing, said method comprising the steps of:
(a) depositing a plurality of nucleic acid molecules onto a surface in discrete, defined locations, wherein at each location is deposited a plurality of first nucleic acid molecules, wherein said first nucleic acid molecules comprise candidate knockout reagents or encode candidate knockout reagents, and a plurality of second nucleic acid molecules, wherein each second nucleic acid molecule comprises (i) a promoter; (ii) a reporter gene comprising a 5′ or 3′ untranslated region, said reporter gene operably linked to said promoter for expression in said cell; and (iii) a target nucleic acid derived from said target gene, said target nucleic acid located within said untranslated region, wherein different first nucleic acid molecules are deposited at different discrete, defined locations;
(b) contacting cells with said nucleic acid molecules under appropriate conditions for entry of the nucleic acid molecules into said cells, whereby said nucleic acid molecules are introduced into the cells in the location in which each of the nucleic acid molecules was deposited;
(c) determining whether a first nucleic acid molecule at a discrete, defined location reduces expression of said reporter gene, relative to expression of said reporter gene in a cell in the absence of said first nucleic acid molecule, wherein reduction of expression of said reporter gene identifies said first nucleic acid molecule at said discrete, defined location as a nucleic acid molecule capable of gene silencing.

2. The method of claim 1, wherein said candidate knockout reagents comprise double-stranded RNA molecules, ribozymes, antisense nucleic acid molecules, or triple helix forming oligonucleotides.

3. The method of claim 1, wherein said reporter gene encodes green fluorescent protein, beta-glucuronidase, luciferase, chloramphenicol transacetylase, beta-galactosidase, red fluorescent protein, beta-lactamase, alkaline phosphatase, or horseradish peroxidase.

4. The method of claim 1, wherein said target gene is located within the 5′ untranslated region of said reporter gene.

5. The method of claim 1, wherein said target gene is located within the 3′ untranslated region of said reporter gene.

6. The method of claim 1, wherein said second nucleic acid molecules further comprise (iv) a polyadenylation sequence located 3′ to said reporter gene.

7. The method of claim 1, wherein said cells are eukaryotic cells.

8. The method of claim 7, wherein said cells are mammalian cells.

9. The method of claim 7, wherein said cells are Drosophila cells.

10. The method of claim 1, wherein said first nucleic acid molecules comprise and/or encode a plurality of different candidate knockout reagents.

11. The method of claim 1, further comprising, between steps (a) and (b), the steps of:
(i) covering said surface with an appropriate amount of a transfection reagent and maintaining the resulting product under conditions appropriate for complex formation between the nucleic acid molecules and the transfection reagent; and
(ii) removing the non-complexed transfection reagent.

12. The method of claim 1, wherein said nucleic acid molecules are components of nucleic acid molecule-containing mixtures, said mixtures further comprising a carrier.

13. The method of claim 12, wherein said nucleic acid molecule-containing mixtures further comprise a buffer that facilitates nucleic acid molecule condensation.

14. The method of claim 12, wherein said nucleic acid molecule-containing mixtures further comprise an appropriate lipid-based transfection reagent.

15. The method of claim 12, wherein said carrier is a gelatin.

16. The method of claim 15, wherein said gelatin is a protein gelatin, a hydrogel, a sugar-based gelatin, or a synthetic gelatin.

17. The method of claim 16, wherein said gelatin is present at a concentration in the nucleic acid molecule-containing mixture ranging from about 0.01% to about 0.5%.

18. The method of claim 17, wherein said gelatin is present at a concentration in the nucleic acid molecule-containing mixture ranging from about 0.1% to about 0.2%.

19. The method of claim 1, wherein said first nucleic acid molecules and/or said second nucleic acid molecules are contained in a vector.

20. The method of claim 19, wherein said vector is an episomal vector or a chromosomally integrated vector.

21. The method of claim 19, wherein said vector is a plasmid or a viral-based vector.

22. The method of claim 1, wherein the surface is glass, polystyrene, or plastic.

23. The method of claim 1, wherein said cells are plated at a density of 0.5×10⁵/cm² to 2.0×10⁵/cm².

24. The method of claim 23, wherein said cells are plated at a density of 0.5×10⁵/cm² to 1.0×10⁵/cm².

25. The method of claim 1, wherein said deposited plurality of nucleic acid molecules in said discrete, defined locations form an array of nucleic acid molecules.

26. The method of claim 25, wherein said array comprises at least 96 different discrete, defined locations of known sequence composition.

27. The method of claim 26, wherein said array comprises at least 192 different discrete, defined locations of known sequence composition.

28. The method of claim 27, wherein the array comprises up to 10,000 to 15,000 different discrete, defined locations of known sequence composition.

29. The method of claim 1, wherein each of said discrete, defined locations is 100-200 μm in diameter.

30. The method of claim 1, wherein each of said discrete, defined locations is 200-500 μm apart from its nearest adjacent discrete, defined location.

31. A method for identifying a nucleic acid molecule capable of gene silencing, said method comprising the steps of:
(a) depositing a plurality of first nucleic acid molecules onto a surface in discrete, defined locations, wherein said first nucleic acid molecule comprise candidate knockout reagents or encode candidate knockout reagents, wherein different first nucleic acid molecules are deposited at different discrete, defined locations;
(b) contacting said nucleic acid molecules with cells expressing a second nucleic acid molecule, wherein said second nucleic acid molecule comprises (i) a promoter; (ii) a reporter gene comprising a 5′ or 3′ untranslated region, said reporter gene operably linked to said promoter for expression in said cell; and (iii) a target nucleic acid derived from said target gene, said target nucleic acid located within said untranslated region, wherein said contacting is performed under appropriate conditions for entry of said first nucleic acid molecules into said cells at the location in which each of the nucleic acid molecules was deposited;
(c) determining whether a first nucleic acid molecule at a discrete, defined location reduces expression of said reporter gene, relative to expression of said reporter gene in a cell in the absence of said first nucleic acid molecule, wherein reduction of expression of said reporter gene identifies said first nucleic acid molecule at said discrete, defined location as a nucleic acid molecule capable of gene silencing.

32. The method of claim 31, wherein said cells are stably transfected with said second nucleic acid molecule.

33. The method of claim 31, wherein said cells are transiently transfected with said second nucleic acid molecule.

34. The method of claim 31, wherein said candidate knockout reagents comprise double-stranded RNA molecules, ribozymes, antisense nucleic acid molecules, or triple helix forming oligonucleotides.

35. The method of claim 31, wherein said reporter gene encodes green fluorescent protein, beta-glucuronidase, luciferase, chloramphenicol transacetylase, beta-galactosidase, red fluorescent protein, beta-lactamase, alkaline phosphatase, or horseradish peroxidase.

36. The method of claim 31, wherein said target gene is located within the 5′ untranslated region of said reporter gene.

37. The method of claim 31, wherein said target gene is located within the 3′ untranslated region of said reporter gene.

38. The method of claim 31, wherein said second nucleic acid molecules further comprise (iv) a polyadenylation sequence located 3′ to said reporter gene.

39. The method of claim 31, wherein said cells are eukaryotic cells.

40. The method of claim 39, wherein said cells are mammalian cells.

41. The method of claim 40, wherein said cells are human or mouse cells.

42. The method of claim 39, wherein said cells are Drosophila cells.

43. The method of claim 31, further comprising, between steps (a) and (b), the steps of:
(i) covering said surface with an appropriate amount of a transfection reagent and maintaining the resulting product under conditions appropriate for complex formation between the nucleic acid molecules and the transfection reagent; and
(ii) removing the non-complexed transfection reagent.

44. The method of claim 31, wherein said nucleic acid molecules are components of nucleic acid molecule-containing mixtures, said mixtures further comprising a carrier.

45. The method of claim 44, wherein said carrier is a gelatin.

46. The method of claim 45, wherein said gelatin is a protein gelatin, a hydrogel, a sugar-based gelatin, or a synthetic gelatin.

47. The method of claim 46, wherein said gelatin is present at a concentration in the nucleic acid molecule-containing mixture ranging from about 0.01% to about 0.5%.

48. The method of claim 47, wherein said gelatin is present at a concentration in the nucleic acid molecule-containing mixture ranging from about 0.1% to about 0.2%.

49. The method of claim 44, wherein said nucleic acid molecule-containing mixtures further comprise a buffer that facilitates nucleic acid molecule condensation.

50. The method of claim 44, wherein said nucleic acid molecule-containing mixtures further comprise an appropriate lipid-based transfection reagent.

51. The method of claim 31, wherein said first nucleic acid molecules and/or said second nucleic acid molecules is contained in a vector.

52. The method of claim 51, wherein said vector is an episomal vector or a chromosomally integrated vector.

53. The method of claim 51, wherein said vector is a plasmid or a viral-based vector.

54. The method of claim 31, wherein the surface is glass, polystyrene, or plastic.

55. The method of claim 31, wherein said cells are plated at a density of 0.5×10⁵/cm² to 2.0×10⁵/cm².

56. The method of claim 55, wherein said cells are plated at a density of 0.5×10⁵/cm² to 1.0×10⁵/cm².

57. The method of claim 31, wherein said deposited plurality of nucleic acid molecules in said discrete, defined locations form an array of nucleic acid molecules.

58. The method of claim 57, wherein said array comprises at least 96 different discrete, defined locations of known sequence composition.

59. The method of claim 58, wherein said array comprises at least 192 different discrete, defined locations of known sequence composition.

60. The method of claim 59, wherein said array comprises up to 10,000 to 15,000 different discrete, defined locations of known sequence composition.

61. The method of claim 31, wherein each of said discrete, defined locations is 100-200 μm in diameter.

62. The method of claim 31, wherein each of said discrete, defined locations is 200-500 μm apart from its nearest adjacent discrete, defined location.

63. A method for identifying a reagent capable of post-transcriptional silencing of a target gene, said method comprising the steps of:
(a) introducing into a cell:
a reagent comprising a double-stranded RNA molecule or a DNA molecule encoding a double-stranded RNA molecule; and an expression vector comprising (i) a promoter; (ii) a reporter gene comprising a 5′ or 3′ untranslated region, said reporter gene operably linked to said promoter for expression in said cell; and (iii) a target nucleic acid derived from said target gene, said target nucleic acid located within said untranslated region; and
(b) determining whether said reagent reduces expression of said reporter gene, relative to expression of said reporter gene in a cell in the absence of said reagent, wherein reduction of expression of said reporter gene identifies said reagent as a reagent capable of post-transcriptional silencing of said target gene.

64. A method for identifying a reagent capable of post-transcriptional silencing of a target gene, said method comprising the steps of:
(a) providing:
(i) a first cell comprising: a reagent comprising a double-stranded RNA molecule or a DNA molecule encoding a double-stranded RNA molecule; and an expression vector comprising a promoter; a reporter gene comprising a 5′ or 3′ untranslated region, said reporter gene operably linked to said promoter for expression in said cell; and a target nucleic acid derived from said target gene, said target nucleic acid located within said untranslated region; and
(ii) a second cell comprising said expression vector but not comprising said candidate reagent; and
(b) determining whether expression of said reporter gene is reduced in said first cell, relative to expression of said reporter gene in said second cell, wherein reduction of expression of said reporter gene in said first cell identifies said reagent as a reagent capable of post-transcriptional silencing of said target gene.

65. A method for identifying a reagent capable of post-transcriptional silencing of a target gene, said method comprising the steps of:
(a) providing:
(i) a first cell comprising: a reagent comprising a double-stranded RNA molecule or a DNA molecule encoding a double-stranded RNA molecule; and a first expression vector comprising a promoter; a reporter gene comprising a 5′ or 3′ untranslated region, said reporter gene operably linked to said promoter for expression in said cell; and a target nucleic acid derived from said target gene, said target nucleic acid located within said untranslated region; and
(ii) a second cell comprising said reagent and a second expression vector comprising said promoter; said reporter gene operably linked to said promoter and not comprising said target nucleic acid; and
(b) determining whether expression of said reporter gene is reduced in said first cell, relative to expression of said reporter gene in said second cell, wherein reduction of expression of said reporter gene in said first cell identifies said reagent as a reagent capable of post-transcriptional silencing of said target gene.

66. The method of claim 65, wherein said reporter gene encodes green fluorescent protein, beta-glucuronidase, luciferase, chloramphenicol transacetylase, beta-galactosidase, red fluorescent protein, beta-lactamase, alkaline phosphatase, or horseradish peroxidase.

67. The method of claim 65, wherein said double-stranded RNA is a small hairpin RNA.

68. An array of nucleic acid molecules, said array comprising a surface having at least 10 different locations, wherein at each location is deposited a plurality of first nucleic acid molecules, wherein said first nucleic acid molecules comprise candidate knockout reagents or encode candidate knockout reagents, and a plurality of second nucleic acid molecules, wherein each second nucleic acid molecule comprises (i) a promoter; (ii) a reporter gene comprising a 5′ or 3′ untranslated region, said reporter gene operably linked to said promoter for expression in said cell; and (iii) a target nucleic acid derived from said target gene, said target nucleic acid located within said untranslated region, wherein different first nucleic acid molecules are deposited at different discrete, defined locations.

69. The array of claim 68, wherein each location is about 100-200 μm in diameter.

70. The array of claim 68, wherein each location is about 200-500 μm from its nearest adjacent location.

71. The array of claim 68, wherein said surface has at least 1000 different locations/cm².

72. The array of claim 71, wherein said surface has at least 10,000 different locations/cm².

73. The array of claim 72, wherein said surface has at least 100,000 different locations/cm².

74. The array of claim 73, wherein said surface has at least 1,000,000 different locations/cm².

75. The array of claim 68, further comprising a plurality of cells on said surface.

76. The array of claim 75, wherein said cells are eukaryotic cells.

77. The array of claim 76, wherein said cells are human, mouse, monkey, or Drosophila cells.

78. The array of claim 75, wherein said cells are at a density of 1×10⁵ cells/cm² to 5×10⁵ cells/cm².

79. The array of claim 68, wherein said candidate knockout reagents comprise double-stranded RNA molecules, ribozymes, antisense nucleic acid molecules, or triple helix forming oligonucleotides.

80. The array of claim 68, wherein said reporter gene encodes green fluorescent protein, beta-glucuronidase, luciferase, chloramphenicol transacetylase, beta-galactosidase, red fluorescent protein, beta-lactamase, alkaline phosphatase, or horseradish peroxidase.

81. The array of claim 68, wherein said target gene is located within the 5′ untranslated region of said reporter gene.

82. The array of claim 68, wherein said target gene is located within the 3′ untranslated region of said reporter gene.

83. The array of claim 68, wherein said second nucleic acid molecules further comprise (iv) a polyadenylation sequence located 3′ to said reporter gene.

84. The array of claim 68, wherein said first nucleic acid molecules comprise and/or encode a plurality of different candidate knockout reagents.


SEQUENCE LISTING (4 sequences)

SEQ ID NO: 1 - DNA, 41 bp, Artificial Sequence
  Synthetic
gaattggctaggcgcggccgctcacatcctggaagaaaggg

SEQ ID NO: 2 - DNA, 46 bp, Artificial Sequence
  Synthetic
ggcgacgtcggagcggccgcgaattcagccagaaacaagttgacgg

SEQ ID NO: 3 - DNA, 46 bp, Artificial Sequence
  Synthetic
ggcgacgtcggagcggccgcgaattcggatgatttgatgctgtccc

SEQ ID NO: 4 - DNA, 41 bp, Artificial Sequence
  Synthetic
gaattggctaggcgcggccgcctttttggacttcaggtggc